(19) United States
(12) Patent Application Publication
(10) Pub. No.: US 2026/0306294 A1
(43) Pub. Date: Oct. 8, 2026

(54) THERMAL TRANSFER PAD WITH PROFILED SURFACE TO REGULATE SKIN TEMPERATURE

(71) Applicant: Chill Therapy, LLC, San Francisco, CA (US)

(72) Inventors: Ursula Ingrid Carney, San Francisco, CA (US); George Carney, San Francisco, CA (US); Paul Maguire, San Francisco, CA (US)

(73) Assignee: Chill Therapy, LLC, San Francisco, CA (US)

(21) Appl. No.: 19/173,082

(22) Filed: Apr. 8, 2025

(51) Int. Cl. A61F 7/02

(52) U.S. Cl. CPC A61F 7/02; A61F 2007/0219; A61F 2007/0222; A61F 2007/0277; A61F 2007/0295

(57) ABSTRACT

A thermal transfer pad that can be cooled or heated and applied to the skin, with a profiled (non-flat) surface that reduces the rate of heat transfer between skin tissue and the pad to better regulate skin temperature. The pad may be applied and left on, compared to existing pads that should be periodically removed and then reapplied. The profiled surface may have protrusions of various patterns, including ridges, pillars, rings, or honeycombs; important parameters that affect performance are the amount of effective transfer surface contacting the skin, and the height of the protrusions. Illustrative ranges of these parameters that may be effective in one or more embodiments of the invention are protrusion heights between 1 mm and 6 mm, and a ratio of effective transfer surface to total application surface between 5% and 40%.

### BACKGROUND OF THE INVENTION

### FIELD OF THE INVENTION

One or more embodiments of the invention are related to the field of thermal pads, which are also known as thermal packs, for heating or cooling tissue. More particularly, but not by way of limitation, one or more embodiments of the invention enable a thermal transfer pad with a profiled surface to regulate skin temperature.

### DESCRIPTION OF THE RELATED ART

Thermal packs known in the art typically have relatively flat surfaces that are applied to the skin. The large contact surface between the pack and the skin leads to rapid skin cooling (for a cold pack) or heating (for a hot pack). As a result, skin temperatures can become uncomfortable or even dangerous unless the pack is periodically removed and then reapplied after a delay. For example, a cold pack that is applied for too long may cause frostbite, and a hot pack that is applied for too long may burn the skin. The protocol for using a thermal pack safely by periodically removing and reapplying is inconvenient for a user, and it may lead to safety concerns if the protocol is not followed correctly. This is inconvenient for a user, and it requires the user to manage their safety which may be a concern if the protocol is not followed correctly or the user is not knowledgeable in matters of frostbite, skin burns, and cold or heat management of the skin and its underlying layers.

For at least the limitations described above there is a need for a thermal transfer pad with a profiled surface to regulate the transfer of cold or heat to the skin, thereby regulating skin temperature.

### BRIEF SUMMARY OF THE INVENTION

One or more embodiments described in the specification are related to a thermal transfer pad with a profiled surface to regulate skin temperature. Embodiments of the invention may reduce the rate of heat transfer between the skin and the pad, thereby causing more gradual and controllable changes in skin temperature compared to traditional flat thermal transfer pads. This reduced rate of heat transfer provides several advantages, including for example greater convenience since the pad need not be cycled on and off or used with a layer between the pad and the skin, increased safety since the skin temperature is less likely to reach dangerous levels (which may cause frostbite or skin burns, for example), and unexpected results including greater therapeutic effectiveness since the pad can be applied continuously for a longer period of time than a traditional flat thermal transfer pad and can be configured with a larger thermal reservoir to last longer compared to a traditional flat thermal transfer pad.

One or more embodiments of the invention may enable a thermal transfer pad with a profiled surface to regulate skin temperature. The thermal transfer pad may include a thermal pad configured to be heated or cooled, with an application surface that is configured to be placed facing a user's skin. There may be protrusions on or extending from the application surface, wherein the application surface includes everything on the portion or side of the pad that faces or is near the user's skin. Each protrusion may have a tip that is configured to be placed against the skin. The tip of each protrusion may have a height, which is the maximum distance between the tip and the application surface. The protrusions may have a thermally conductive material that transfers heat between the thermal pad and the skin. The application surface may be partitioned into conductive zones from which the protrusions protrude, and air channel zones that do not contact the skin when tips of the protrusions are placed against the skin. The height of each protrusion may be between 1 mm and 6 mm, inclusive. The protrusions may have an effective transfer surface that is the orthogonal projection of the tips of the protrusions onto the application surface. The surface area of the effective transfer surface may be between 5% and 40%, inclusive, of the surface area of the application surface.

One or more embodiments of the thermal transfer pad may be configured to transfer heat from the skin to the thermal transfer pad. One or more embodiments of the thermal transfer pad may be configured to transfer heat from the thermal transfer pad to the skin.

In one or more embodiments, in each square subregion of the application surface with an area of 16 square centimeters, the surface area of the intersection of the effective transfer surface with the square subregion may be between 5% and 40%, inclusive, of the surface area of the square subregion.

In one or more embodiments, the tips of the protrusions may be substantially flat.

In one or more embodiments, the protrusions may be ridges, and the width of each ridge may be between 0.5 mm and 10.0 mm, inclusive. Each air channel zone between adjacent ridges may have a width that is less than or equal to three times the maximum distance between the tips of the ridges and the application surface.

In one or more embodiments, the protrusions may be pillars. The orthogonal projection of the tip of each pillar onto the application surface may have an area between 1 square millimeter and 100 square millimeters, inclusive.

In one or more embodiments, the protrusions may have a hardness between 0 and 80 Shore D, inclusive.

In one or more embodiments, the protrusions may contain a compliant material. This compliant material may be an elastomer, for example. The elastomer may be silicone rubber, or any biocompatible elastomer for example.

In one or more embodiments, the thermal conductivity of the protrusions material may be between 0.14 W/m-K and 4.3 W/m-K, inclusive.

In one or more embodiments, where the height of the protrusions is h, the application surface total surface area is S, the effective transfer surface total surface area is A, the product (A/S)(1/h) may be between 0.01 mm⁻¹ and 0.40 mm⁻¹, inclusive.

In one or more embodiments, the thermal pad may be filled with a liquid material. In one or more embodiments, the liquid material may include a gel. In one or more embodiments, the liquid material may have a freezing point at or below-20 degrees Fahrenheit.

In one or more embodiments, the thermal transfer pad may be filled with a solid material. In one or more embodiments, the solid material may include an aggregate.

In one or more embodiments, the profiled surface of the thermal transfer pad may occupy between 10% and 100% of the total surface area of the top and bottom of the pad.

### BRIEF DESCRIPTION OF THE DRAWINGS

The above and other aspects, features and advantages of the invention will be more apparent from the following more particular description thereof, presented in conjunction with the following drawings wherein:

FIG. 1A shows a typical thermal transfer pad in the prior art, with a substantially flat surface that is applied to the skin, and it illustrates that such a pad may rapidly reduce skin temperature to a potentially dangerous level.

FIG. 1B shows a typical use case for a thermal transfer pad in the prior art, where it is recommended to apply the pad for only short periods of time to avoid excessive cooling.

FIG. 2A shows an illustrative embodiment of the invention: a thermal transfer pad with a profiled surface that avoids the issues illustrated in FIG. 1A and FIG. 1B.

FIG. 2B shows a vertical cross-section of the thermal transfer pad of FIG. 2A, illustrating the profile of protrusions that extend from the pad's application surface, and the air gaps between protrusions.

FIG. 2C shows horizontal sections of the thermal transfer pad of FIG. 2A, illustrating protrusions formed as wavy ridges, and a substantially flat application surface below the protrusions.

FIG. 2D shows the vertical cross-section of FIG. 2B, labelling parameters that may affect the thermal transfer rate from the pad to the user's skin.

FIG. 2E shows an illustrative thermal transfer pad with a profiled region of the pad's surface and other regions that are substantially flat.

FIG. 3 illustrates application of the thermal transfer pad of FIG. 2A to a user's skin; the air gaps reduce the rate of heat transfer to the skin and prevent the skin temperature from reaching dangerous levels such as those shown in FIG. 1A.

FIG. 4 shows a theoretical heat transfer model that identifies key parameters that affect the rate of skin temperature change: the ratio of the surface area touching the skin to the total treated skin surface, and the reciprocal of the height of the protrusions connecting the thermal transfer pad to the skin.

FIG. 5 shows top views of six illustrative shapes of protrusions that may be used in one or more embodiments of the invention.

FIG. 6 shows vertical cross-section views of eight illustrative protrusion profiles that may be used in one or more embodiments of the invention.

FIG. 7 shows an application surface with four illustrative protrusions of different shapes, and it illustrates calculation of the effective transfer surface associated with the tips of the protrusions that contact the skin.

FIG. 8 shows the profiles of FIG. 6 with indications of the protrusion tips and the effective transfer surfaces associated with these tips.

FIG. 9 shows a top view of an embodiment of the invention with protrusions consisting of wavy ridges; it illustrates that the effective transfer surface ratio is relatively constant across the application surface.

FIG. 10A illustrates a potential issue when protrusions are too short or the distance between protrusions is too great: the skin may touch the air channels and thereby reduce the effectiveness of the profiled surface by allowing excess skin contact.

FIG. 10B illustrates a potential issue when protrusions are too long: the protrusions may collapse and increase the effective heat transfer surface.

FIG. 11 shows the dimensions of an illustrative profile for an embodiment of the invention.

FIG. 12 shows results of an experiment comparing temperature changes with a thermal transfer pad using the profiled surface of FIG. 11 to temperature changes with a thermal transfer pad with a flat surface; this experiment illustrates that the profiled surface prevents excessive cooling.

### DETAILED DESCRIPTION OF THE INVENTION

A thermal transfer pad with a profiled surface to regulate skin temperature will now be described. In the following exemplary description, numerous specific details are set forth in order to provide a more thorough understanding of embodiments of the invention. It will be apparent, however, to an artisan of ordinary skill that the present invention may be practiced without incorporating all aspects of the specific details described herein. In other instances, specific features, quantities, or measurements well known to those of ordinary skill in the art have not been described in detail so as not to obscure the invention. Readers should note that although examples of the invention are set forth herein, the claims, and the full scope of any equivalents, are what define the metes and bounds of the invention.

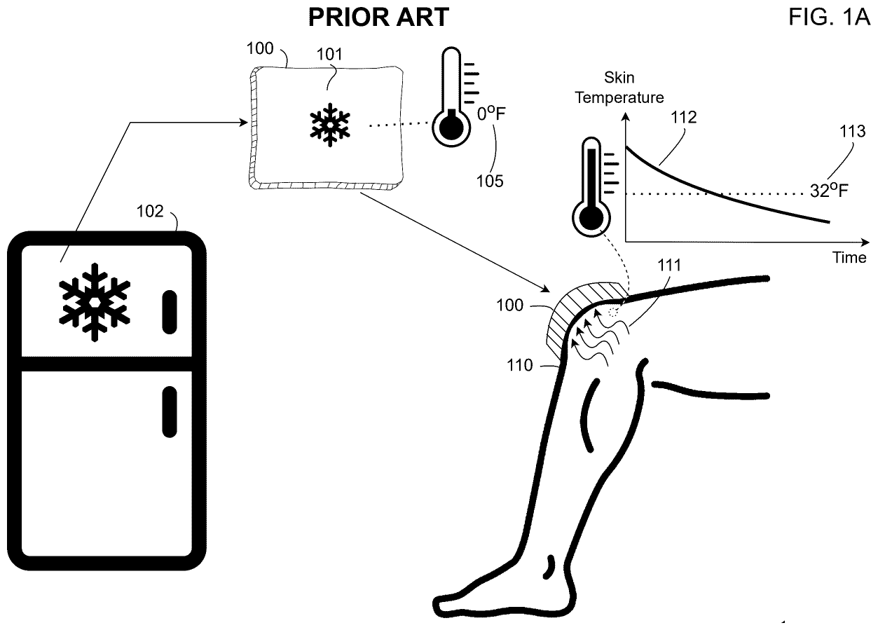

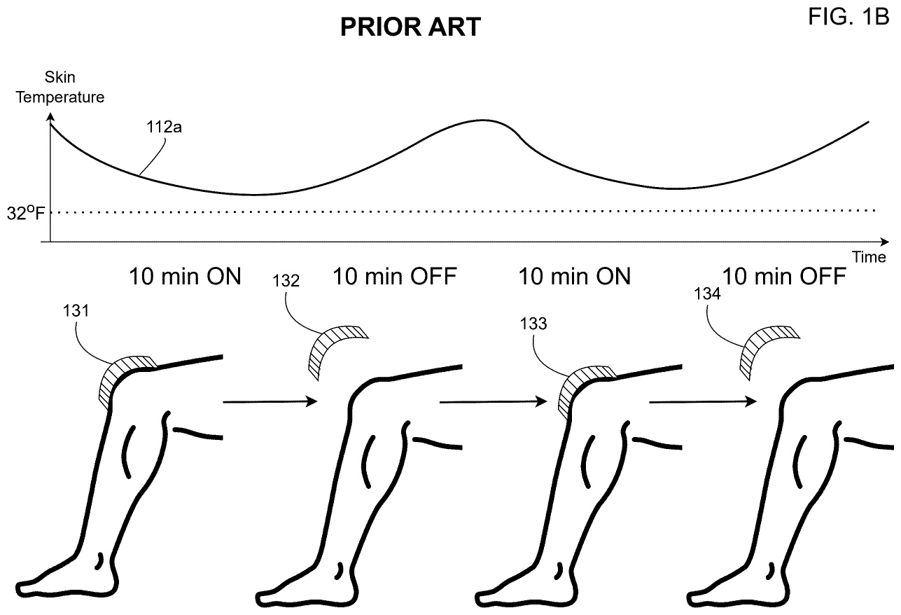

Thermal transfer pads may be used to warm or cool tissues, for example to aid in injury recovery. For example, gel-filled packs may be cooled in a freezer or heated in a microwave, and then applied to a site on a person's body. FIG. 1A illustrates use of a typical thermal transfer pad 100 found in the existing art. Thermal transfer pad 100, which may for example be a silicone shell filled with a gel material, may be placed in freezer 102 and then removed and applied to a site 110, such as a knee or other body part. (FIG. 1A and subsequent examples illustrate use of thermal transfer pads for cooling; similar or identical pads may be used for heating, and the benefits of embodiments of the invention apply to either cooling or heating applications.) For thermal transfer pads known in the art, the surface 101 that is applied to the skin is generally flat or substantially flat, and it is flexible so that it conforms closely to the skin even when applied to a curved body part such as a knee. FIG. 1A illustrates a drawback to this existing design. In freezer 102, thermal transfer pad 100 is cooled to a typical freezer temperature 105 of approximately 0° F. When flat surface 101 of pad 100 is applied to the skin, heat flow 111 occurs rapidly from the tissue under the pad to the pad, because the contact surface between the pad and the skin is large. This leads to a rapidly decreasing skin temperature 112 under the pad, as the skin temperature approaches the temperature 105 of the pad. Skin temperature 112 may fall below freezing temperature 113 relatively quickly, which may cause injury such as frostbite. (Similarly a thermal transfer pad used for heating may cause skin temperature to rise quickly, which may cause injury such as burning of the skin.) As described below, embodiments of the invention use a novel design to avoid this situation.

Because of the issue shown in FIG. 1A, users of thermal transfer pads are cautioned to apply them only for a limited period of time, or to put a layer between the pad and the skin. Both of these solutions make use of a thermal transfer pad less convenient, and they risk injury due to noncompliance with these recommendations. FIG. 1B illustrates a typical workaround for the problem of excessive and overly rapid cooling: a user alternates between periods of putting on or taking off the thermal transfer pad, such as on 131 for 10 minutes, followed by off 132 for 10 minutes, followed by on 133 for 10 minutes, followed by on 134 for 10 minutes, etc. With this protocol, the skin temperature 112a may, in some cases, not fall to dangerous levels. Embodiments of the invention are configured to prevent this potentially dangerous situation without the complex on/off protocol illustrated in FIG. 1B. If instead of alternating on/off periods, a user places a layer between the pad and the skin, the therapeutic effect of the pad may vary greatly depending on the material and thickness selected for the layer. This workaround therefore may result in variable and inconsistent therapeutic effects. For example, if one user chooses a thin layer of satin to place between the pad and the skin, and a different user chooses a terry-cloth towel, they will experience very different therapeutic effects because the different fabrics restrict heat flow by different amounts. As described below, embodiments of the invention overcome this variability, inconsistency, and inconvenience by not requiring a layer between the pad and the skin.

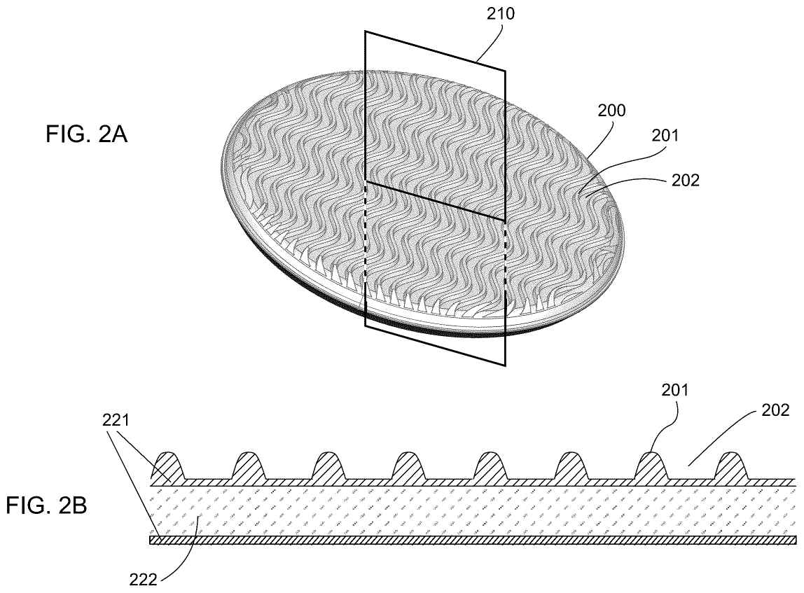

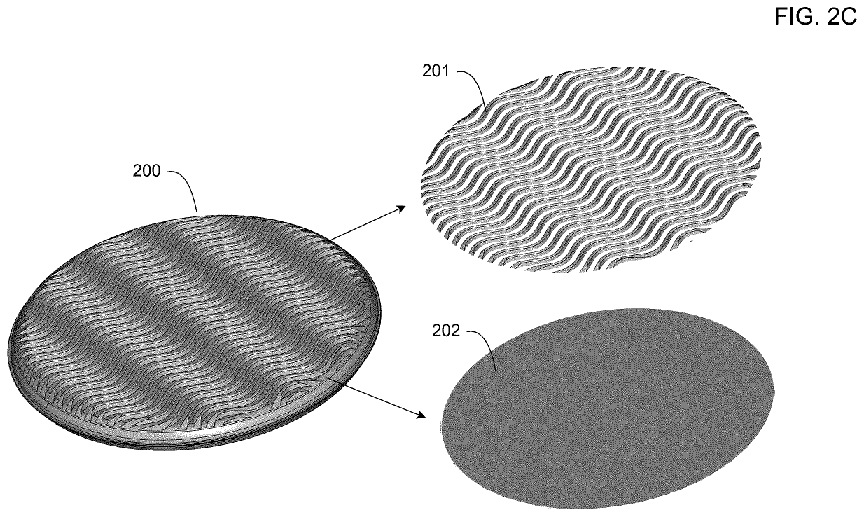

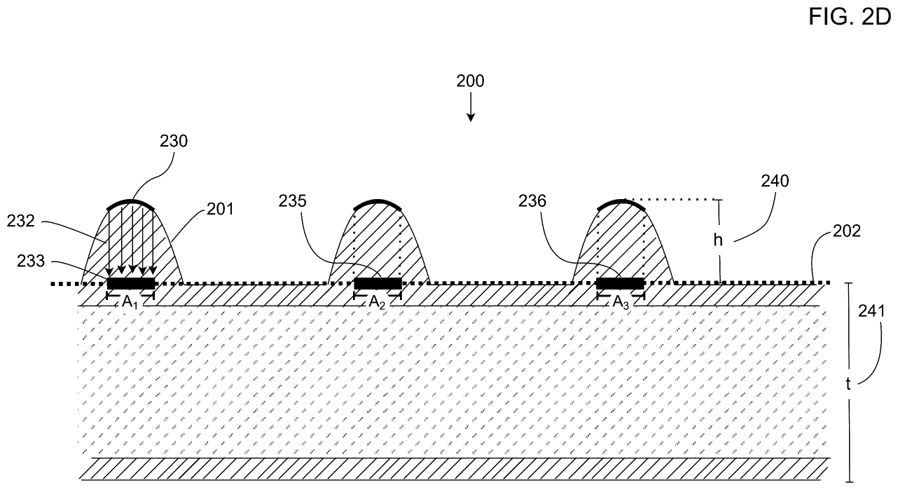

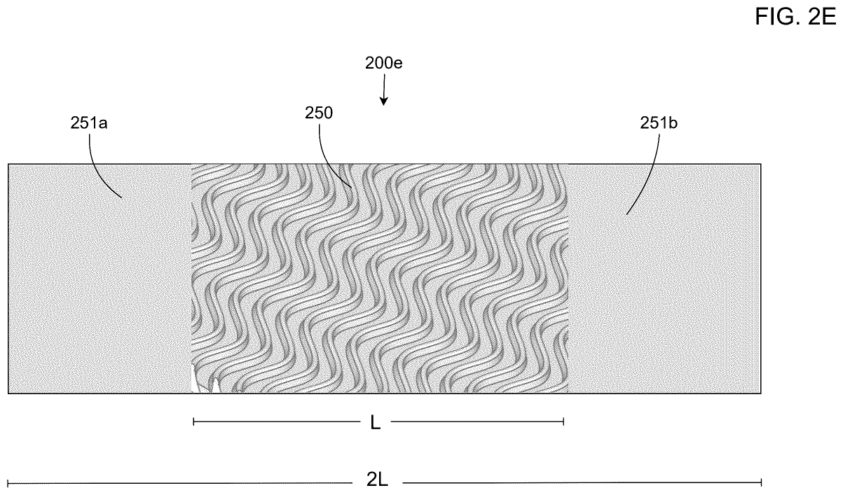

FIG. 2A shows an illustrative embodiment 200 of the invention, which is a thermal transfer pad with a profiled (non-flat) surface. FIG. 2A shows the top side of the pad, which is the side that is applied against the user's skin. The bottom side may be flat, or it may also be profiled (possibly with a different pattern) in one or more embodiments so that either side can be placed against the skin. In this embodiment, the shell of the pad enclosing gel or another material that can be cooled (or heated or both) has protrusions such as ridge 201 and air gaps 202 between ridges. The ridges in this illustrative embodiment have a wavy shape; other embodiments may have ridges or other types of protrusions of any shapes and patterns. A protrusion may be any portion of the thermal transfer pad that is elevated above a substantially flat surface that encloses the thermal reservoir material. Protrusions may be of any shape, size, and extent. A ridge may be for example a protrusion that is substantially longer in one direction along this flat surface than it is wide. Protrusions need not be ridges; for example, some protrusions may be pillar-shaped in that their extent along the flat surface is substantially similar in different directions. FIG. 2B shows a vertical cross section of pad 200 along plane 210. The cross-hatched top and bottom of the pad may for example be made of a compliant material 221, such as silicone rubber or another elastomer for example. The middle 222 may be made of any material or materials that can be heated or cooled for the intended application of the thermal transfer pad. These materials may be liquid, solid, or a combination thereof. Illustrative materials may include for example, without limitation, a gel, or an aggregate of many individual solid items such as granules or beads of any sizes or shapes. In one or more embodiments, the materials used for the protrusions (such as the ridges in pad 200) may or may not be identical to those used in the top and bottom surfaces that enclose the gel (or similar) interior. Typically, but not necessarily, the material that contacts the skin (including the tips of the protrusions) may be compliant to improve comfort, and the materials of the pad may be flexible so that the pad can conform to different body parts. The Shore D durometer of the protrusions, for example, may be between 0 and 80, inclusive, in one or more embodiments. (Hardness may be measured, for example, using the standardized hardness test ASTM D2240-15(2021).) Since the pad may be cooled for example in a freezer, in a pad with a liquid interior (such as a gel), this liquid interior material may be selected in one or more embodiments to not freeze solid at temperatures at or below 0 degrees F. For example, in one or more embodiments a liquid interior may have a freezing point at or below −20 degrees F for example.

FIG. 2C shows two horizontal cross-sections of thermal transfer pad 200: the “application surface” 202, which is the top of the enclosing layer on the side of the pad that is applied to the skin, and the protrusions 201 that extend upward from application surface 202. Typically, but not necessarily, the application surface 202 is relatively flat, and the protrusions 201 create the final profiled surface of the complete thermal transfer pad. Protrusions extend from only a portion of the application surface, which we call “conductive zones” since heat transfer occurs between the pad and the skin through these zones; the remainder of the application surface is “air channel zones” from which protrusions do not extend.

FIG. 2D annotates the vertical cross section view of FIG. 2B to illustrate areas and dimensions of interest in the subsequent specification. Dotted line 202 shows the application surface of thermal transfer pad 200. Protrusions such as protrusion 201 extend from application surface 202. Each protrusion has a tip that will contact the skin when the thermal transfer pad is applied to the user's body. The tips are indicated with thicker lines; for example, protrusion 201 has tip 230. Protrusion height 240 is the maximum distance of the protrusion tips from application surface 202. Thickness 241 is the total thickness of the pad, not including the protrusions. In the following specification we will refer to the “effective transfer surface” of the pad (or of a portion of the pad); this is defined as the area of the orthogonal projection of the protrusion tips onto the application surface. For example, area 233 is the effective transfer surface of tip 230 of protrusion 201, via orthogonal projection 232 of region 230 onto application surface 202. Similarly, areas 235 and 236 are the effective transfer surfaces associated with the other protrusions visible in FIG. 2A. The total effective transfer surface of a thermal transfer pad is the sum of the areas of the tip projections onto the application surface for all of the pad's protrusions, which would include areas A₁+A₂+A₃ for the protrusions shown in FIG. 2D.

One or more embodiments of the invention may have portions of the thermal transfer pad with substantially flat surfaces in addition to the portions with profiled surfaces. For example, FIG. 2B shows that the bottom surface of pad 200 is substantially flat, and the top surface is profiled. Users may therefore select either the top surface or the bottom surface for application, depending on the desired thermal transfer characteristics for a particular application. Moreover, in some applications it may be desirable to have different thermal transfer characteristics on different areas of a person's body, and as a result different regions of a thermal transfer pad may have different types of surfaces. FIG. 2E shows a top view of an illustrative thermal transfer pad 200e that has a center region 250 with a profiled surface and left and right side regions 251a and 251b with substantially flat surfaces. In this illustrative pad, the surface area of profiled center region 250 is half the total surface area of the top of the pad. Embodiments may have a profiled region (or multiple profiled regions) that occupies any desired fraction of the total surface of the pad. For example, if the bottom side of pad 200e is substantially flat, then the profiled region 250 represents 25% of the total surface area (top and bottom sides) of the pad. The fraction of the total pad surface area occupied by profiled regions may for example be between 10% and 100% in illustrative embodiments of the invention.

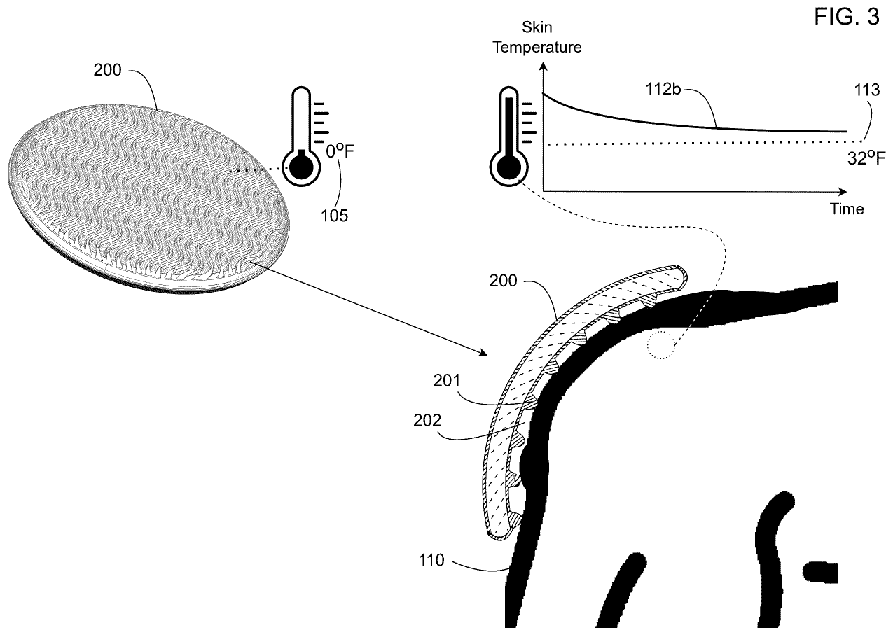

FIG. 3 illustrates application of embodiment 200 to the user's skin at site 110. In contrast to the situation in FIG. 1A with a thermal transfer pad having a flat surface, thermal transfer pad 200 contacts the skin at the protrusion tips, leaving air gaps such as gap 202 between the skin and the body of the thermal transfer pad. This feature slows the rate of thermal transfer between the tissue and the thermal transfer pad. As a result, even if pad 200 is cooled to freezer temperature 105, the skin temperature 112b does not go below freezing temperature 113. It is therefore unnecessary to use complicated protocols such as that shown in FIG. 1B, or protocols that place a layer between the pad and the skin (with potential variability due to different layer choices), to prevent tissue damage. Embodiments of the invention therefore enhance user convenience and safety compared to thermal transfer pads in the prior art. Another advantage of embodiments of the invention over the prior art is increased therapeutic effectiveness, since the thermal transfer pad can be left on the skin for an extended period of time, unlike the situation for existing pads illustrated in FIG. 1B, where a pad with a flat surface may be cycled on and off periodically to prevent over cooling. Since embodiments of the invention slow the rate of thermal transfer, they can be applied continuously for an extended period of time, increasing the effectiveness (and convenience) of therapy by keeping skin temperature in a desirable range for a longer time. An additional advantage of embodiments of the invention over the prior art is that embodiments may be configured to have greater volumes of thermal reservoir material, thereby allowing treatment for a longer duration. Existing pads known in the art typically are relatively thin (with low volumes of gel), to reduce the risk of over-cooling the skin (or over-heating when used as heating pads). As a result, users need to either frequently re-cool (or re-heat) their pads before applying again, or to stock and use multiple pads in succession. With embodiments of the invention, the slower thermal transfer between the pad and the skin allows the pad to be substantially thicker (with a greater volume of gel or other thermal reservoir material) without risking injury. This again increases user convenience and therapeutic effectiveness.

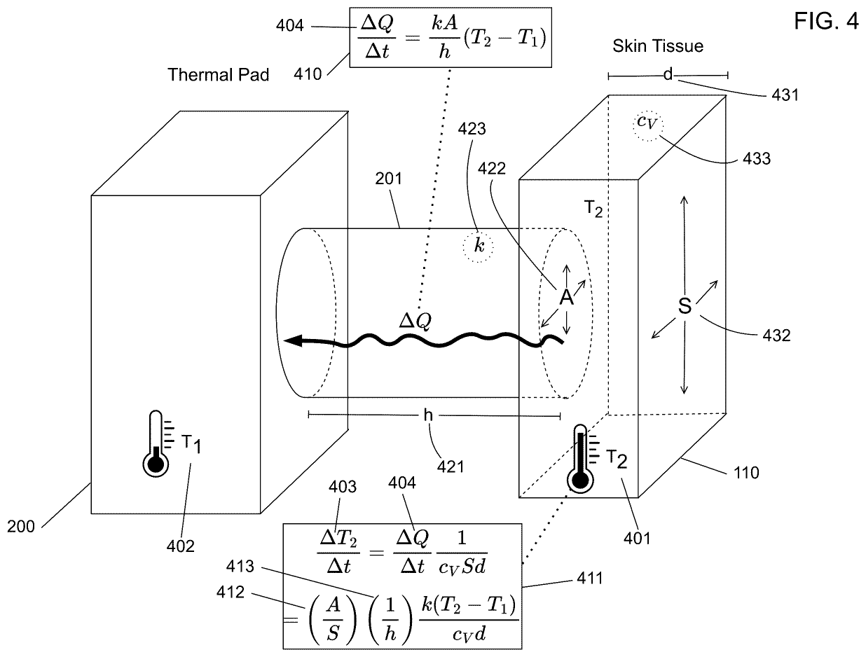

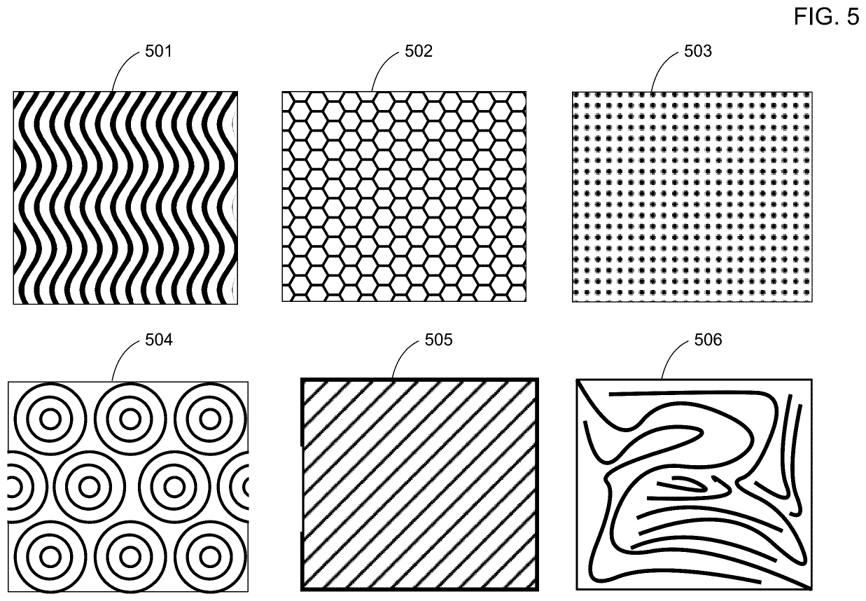

FIG. 4 shows a simplified theoretical thermal transfer model that provides insight into the parameters that influence the effectiveness of embodiments of the invention in slowing tissue temperature changes. Thermal transfer pad 200 is modeled as a heat reservoir at temperature 402 (which may be freezer temperature for example, as illustrated in FIG. 3, for a pad that is used for cooling). Skin tissue 110 is modeled as a volume of surface area 432 and depth 431, with a uniform volumetric heat capacity 433. The temperature 401 of the skin tissue changes over time at a rate 403. (In this highly simplified model, we assume that temperature is uniform throughout the skin tissue volume 110.) A physical “pipe” 201, which models the collection of protrusions of thermal transfer pad 200, connects skin tissue 110 and pad 200. Heat flows through this pipe 201 from the skin tissue to the pad (or vice-versa if the thermal transfer pad is used for heating instead of cooling). Transfer of heat may occur in either direction: from the skin tissue to the pad for cooling pad applications, and from the pad to the skin tissue for heating pad applications. Pipe 201 has a surface area 422, which corresponds to the effective transfer surfaces of the protrusions, and a height 421, which corresponds to the protrusion heights. (The “pipe” is shown as a cylinder for convenience, but it could be of any shape, and it could be a combination of multiple separate connections.) The pipe material has thermal conductivity 423. With these parameters, the rate of heat flow (thermal transfer) 404 between the skin tissue and the thermal transfer pad (in either direction) is modeled in equation 410, which is essentially Newton's law of cooling. The rate of skin temperature change 403 is then given by equation 411. This equation shows that the temperature change rate depends on the ratio 412 of the effective transfer surface 422 to the total treatment surface 432, and it depends inversely on the height 421 of the protrusions. Reducing the rate of thermal transfer, and thus of skin temperature change, can therefore be accomplished by reducing the density of protrusions, by making protrusions taller, or both. The thermal conductivity 423 of the protrusions may also effect the rates of thermal transfer and temperature change; in one or more embodiments this conductivity may for example be between 0.14 W/m-K and 4.3 W/m-K, inclusive.

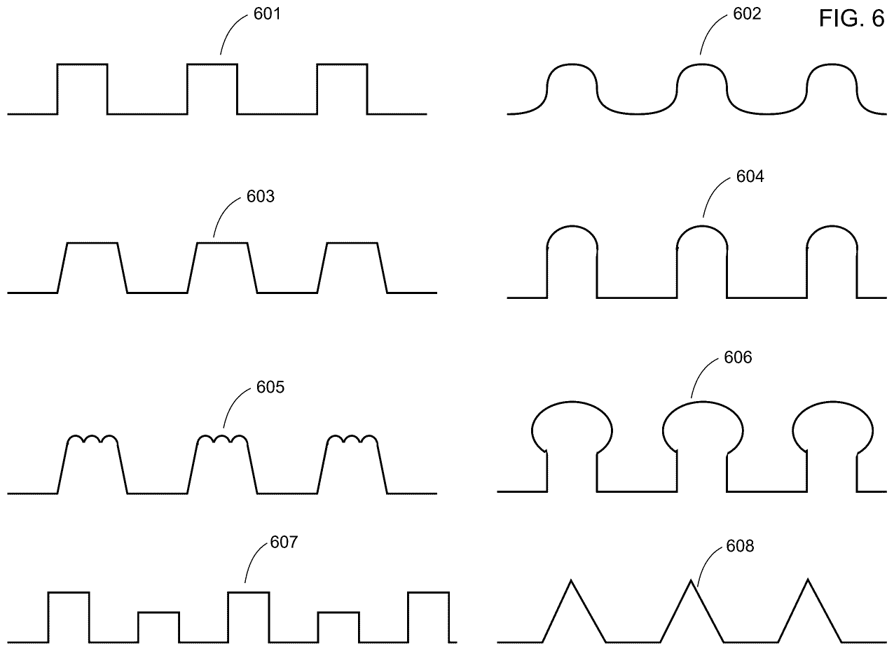

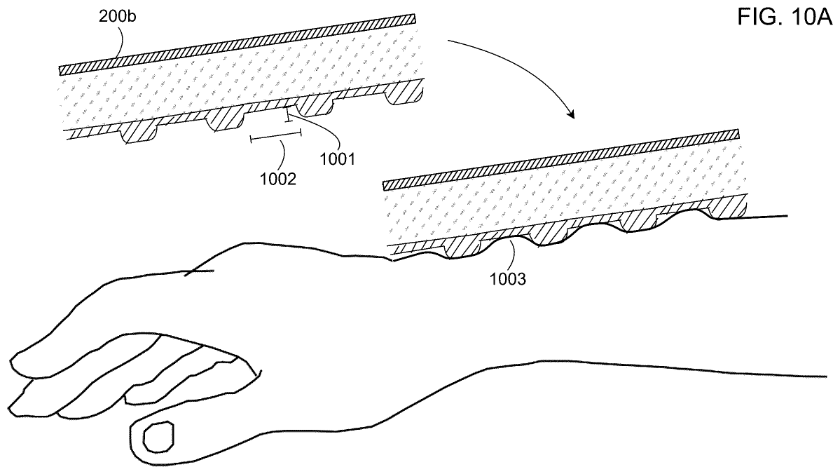

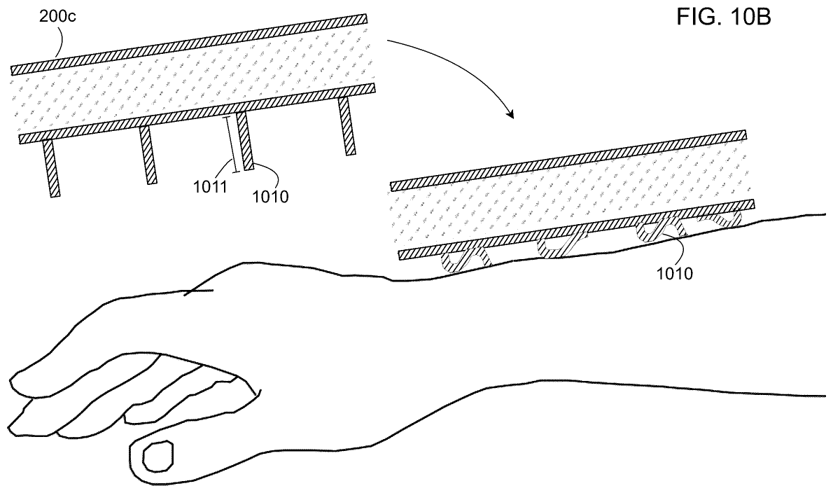

According to the model of FIG. 4, the precise shape of protrusions may be less critical than the total density of the protrusions across the application surface of the thermal transfer pad. Embodiments of the invention may therefore have protrusions of various shapes and patterns, as illustrated in FIG. 5 for example. FIG. 5 shows top views of various protrusion patterns, with protrusions indicated in black and the air channels indicated in white. These patterns are illustrative; one or more embodiments of the invention may have any protrusion patterns that provide desired thermal transfer characteristics. (As illustrated below in FIGS. 10A and 10B, there may be additional considerations in the selection of protrusion patterns, such as minimum or maximum protrusion heights or inter-protrusion spacings that may affect the effectiveness of the thermal transfer pad.) Protrusions may be for example, without limitation: ridges that are straight, curved, irregular, bent, swirled, or circular; pillars that are cylindrical, square, pyramidal, or polygonal; honeycomb patterns; crosshatches of any shapes and angles; or mixtures of any of these patterns. Illustrative protrusion patterns shown in FIG. 5 are the wavy ridges 501 of embodiment 200, a honeycomb of ridges 502; cylindrical pillars 503; concentric circular ridges 504; diagonal ridges 505; and irregularly shaped ridges 506.

Similarly, the profile of protrusions may have various shapes. FIG. 6 shows illustrative protrusion profiles (vertical cross-sections) that may be used in one or more embodiments. Profile 601 corresponds to ridges or pillars that are rectangular; profile 602 shows curved protrusions; profile 603 corresponds to tapered protrusions that have flat tips and are wider at their bases; profile 604 corresponds to rectangular protrusions with curved (hemispherical) tips; profile 605 is a variation of profile 603 with curved tips instead of flat tips; and profile 606 shows “mushroom shaped” tips. One or more embodiments of the invention may have ridges or pillars (or other protrusion features) of different heights; profile 607 shows an example with alternating heights where a lower protrusion is between each higher protrusion. Embodiments may have protrusions with any number of different heights arranged in any pattern, not necessarily alternating as in profile 607. One or more embodiments of the invention may have some or all tips with sharp or pointed tips; profile 608 shows an illustrative profile with triangular protrusions that have sharp tips.

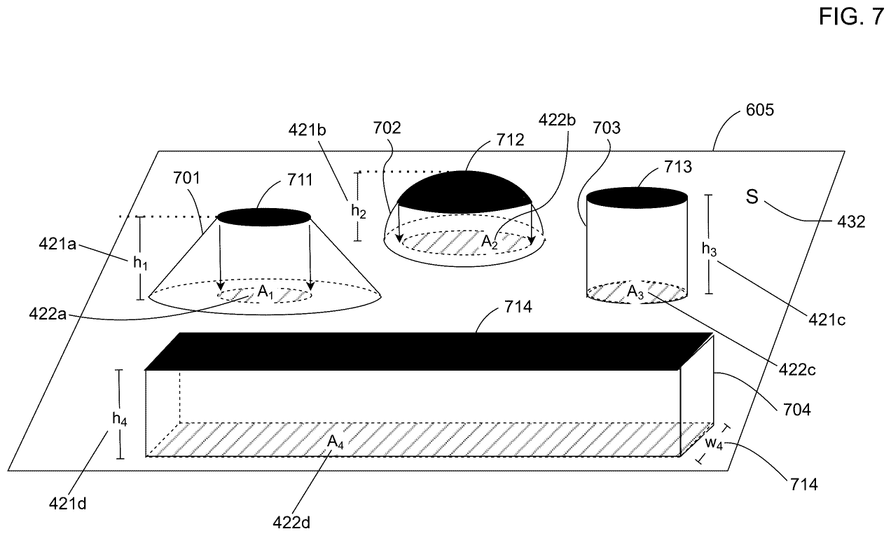

FIG. 7 shows an illustrative application surface 605 with four differently shaped protrusions. The tip of each protrusion is colored black, and the orthogonal projection of each tip onto the application surface is hatched. Conical protrusion 701 has a flat tip 711; hemispherical protrusion 702 has a curved tip 712; cylindrical protrusion 703 has a flat tip 713; and rectangular protrusion 704 has a flat tip 714. The orthogonal projections of tips 711, 712, 713, and 714 onto application surface 605 are 422a, 422b, 422c, and 422d, respectively. The sum of the areas 422a, 422b, 422c, and 422d are the effective transfer surface of the thermal transfer pad, and the ratio of this sum to the application surface area 432 corresponds to factor 412 in equation 411 of FIG. 4. Protrusions 701, 702, 703, and 704 have heights 421a, 421b, 421c, and 421d, respectively. The reciprocal of the maximum (or average) of these heights corresponds to factor 413 in equation 411 of FIG. 4. Protrusions 701, 702, and 703 may be considered “pillars” because they are not substantially extended along a horizontal axis along the application surface, while protrusion 704 may be considered a “ridge”. Some protrusions may be neither ridges nor pillars, and some protrusions may share elements of both ridges and pillars. Pillars, ridges, and other protrusions may be of any shape and size. In one or more embodiments, each pillar may have an associated effective transfer surface (such as 422a, 422 b, 422c) of between 1 square millimeter and 100 square millimeters, inclusive. In one or more embodiments, each ridge may have a width 714 (along the horizontal plane on the short axis of the ridge) between 0.5 millimeters and 10.0 millimeters, inclusive.

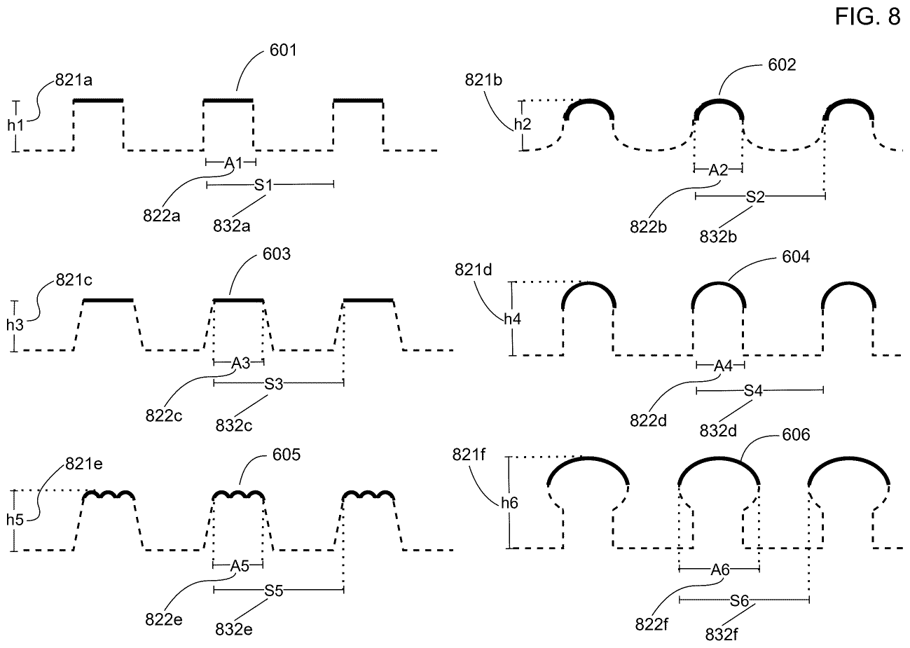

FIG. 8 illustrates the factors 412 (effective transfer surface divided by application surface area) and 413 (reciprocal of protrusion height) for the protrusion profiles of FIG. 6. The tips of the protrusions are shown in thicker lines. In this example we assume for simplicity that the protrusions are ridges that extend across the entire application surface at a constant density. This assumption implies that the protrusion pattern is periodic, and that therefore the ratio of the effective transfer surface for all protrusions to the total application surface area is equal to the effective transfer surface for a single protrusion divided by the area of the “period,” which is the spacing between the start of a protrusion tip and the start of the subsequent protrusion tip. The heights of protrusions 601, 602, 603, 604, 605 and 606 are 821a, 821b, 821c, 821d, 821e, and 821f, respectively. The effective transfer surface areas of a single protrusion (ridge) are 822a, 822b, 822c, 822d, 822e, and 822f, respectively; and the surface areas of a “period” are 832a, 832b, 832c, 832d, 832e, and 832f. Therefore, the product of factors 412 and 413 from equation 411 of FIG. 4 for these profiles are respectively 822a divided by 832a times the reciprocal of 821a, 822b divided by 832b times the reciprocal of 821b, 822c divided by 832c times the reciprocal of 821c, 822d divided by 832d times the reciprocal of 821d, 822e divided by 832e times the reciprocal of 821e, and 822f divided by 832f times the reciprocal of 821f. The protrusion parameters (height, effective transfer surface, and spacing) may be selected so that this product is within a desired range. For example, in one or more embodiments of the invention, this product may be between 0.01 and 0.40 mm⁻¹, inclusive.

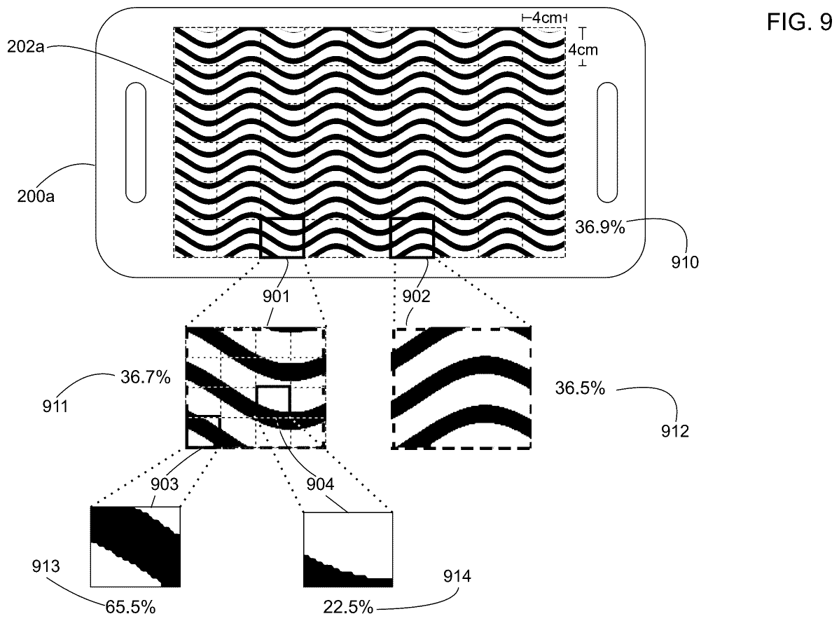

Although the ratio of the total effective transfer surface (the area of the projected tips) to the total application surface is an important parameter that affects the rate of temperature change, it may also be important that this ratio be relatively constant across the application surface, so that skin cooling (or heating) is relatively uniform across the area where the pad is applied. FIG. 9 illustrates a thermal transfer pad 200a with an application surface 202a; the black wavy lines represent the projections of the tips of the protrusions (the effective transfer surface), which in this example are ridges, and the white areas represent air gaps between the protrusions. The ratio 910 of the effective transfer surface (the black region within 202a) and the total surface area 202a, is 36.9%. (In one or more embodiments, this ratio may be for example between 5% and 40%, inclusive.) Moreover, the pattern of protrusions across the application surface 202a makes this ratio relatively constant within subregions of 202a. FIG. 9 illustrates a grid of squares 4 cm by 4 cm in size applied over the application surface 202a; the grid is shown with dotted lines. In each 16 cm² square of this grid, the ratio of the effective transfer surface within the square (i.e., the intersection of the total effective transfer surface with the square) to the area of the square (16 cm²) is close to the overall ratio 910. For example, in square 901 this ratio 911 is 36.7%, and in square 902 this ratio 912 is 36.5%. Clearly if subdivision continues to extremely small grid regions, the ratio of effective transfer surface to grid region area cannot remain constant; for example, if square 901 is further subdivided, illustrative subdivided squares 903 and 904 have very different ratios 913 and 914. Relatively constant ratio of effective transfer surface to application surface area to grid squares down to 16 cm² is sufficient to provide constant cooling or heating of the skin across the application area.

Turning now to the issue of protrusion height, in addition to the effect of the factor 413 (reciprocal of height) on temperature change, there are practical considerations that may limit the effective range of protrusion height in embodiments of the thermal transfer pad. For example, as shown in FIG. 10A for pad 200b, if protrusion height 1001 is too short, and if width 1002 of air gaps between protrusions is too wide, then the pad may be ineffective because the user's skin may deform to touch the air gaps when the pad is applied, for example at a location 1003 between protrusions. Conversely, FIG. 10B illustrates a potential problem if protrusions are too tall or too narrow: thermal transfer pad 200c has very tall protrusions such as 1010 with a height 1011; when the pad is applied, protrusions may collapse (since they may be made of a flexible material such as an elastomer), leading to multiple contact points such as location 1010 between the protrusion material and the skin, rather than just on the protrusion tips as intended. This may increase the thermal transfer rate and reduce the effectiveness of the profiled surface. The inventors have experimented with various protrusion heights and profiles and has found that in some configurations a protrusion height between 1 mm and 6 mm, and a protrusion width between 0.5 mm and 10 mm, inclusive, are effective. Moreover, the inventors have found that in one or more embodiments it is effective to have the width of each air channel zone between adjacent protrusions be less than or equal to three times the height of the protrusions.

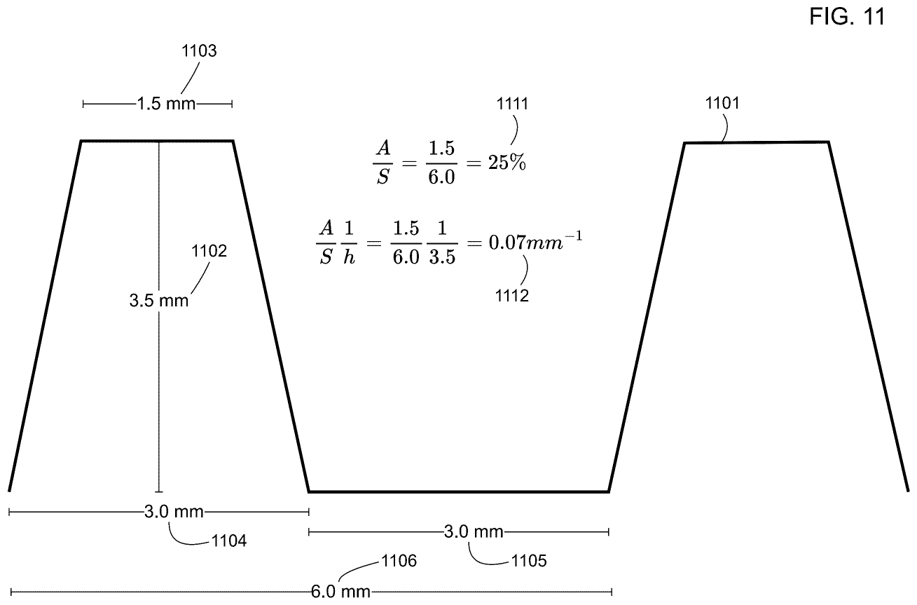

FIG. 11 shows and illustrative profile 1101 for ridges that may be used in one or more embodiments of the invention. This profile has ridges with flat tips of width 1103, with bases of width 1104; in this example the tip width 1103 of 1.5 mm is half the base width 1104 of 3.0 mm. (Having a wider protrusion base than tip may make protrusions less likely to collapse in some situations.) Protrusion height 1102 is 3.5 mm. Air channel zones between adjacent ridges have width 1105 of 3.0 mm; therefore, the width of a “period” 1106 is 1104 plus 1105, 6 mm. With these illustrative dimensions, the ratio of effective transfer surface area to application surface area 1111 is 25%, and the product 1112 of this ratio with the reciprocal of protrusion height is 0.07 mm⁻¹. Profile 1101 and the dimensions shown in FIG. 11 are illustrative; one or more embodiments of the invention may use different shapes and dimensions. In one or more embodiments, the protrusion height 1102 may be for example, without limitation, between 1 mm and 6 mm, inclusive; the ratio 1111 of effective transfer surface to application surface area may be for example, without limitation, between 5% and 40%; and the product 1112 of this ratio with the reciprocal of protrusion height may be for example, without limitation, between 0.01 mm⁻¹ and 0.40 mm⁻¹, inclusive.

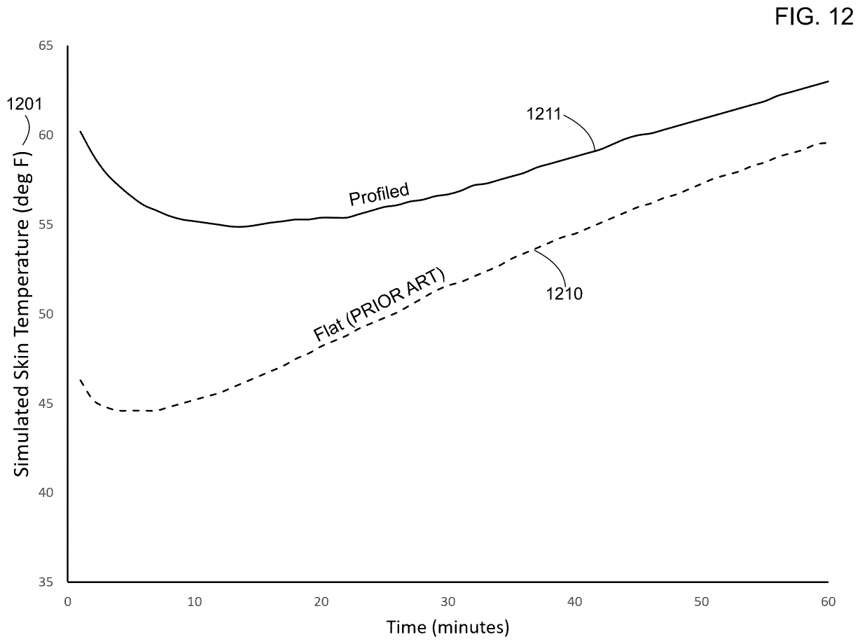

FIG. 12 shows the results of an experiment performed by the inventors that demonstrates the profiled surface 1101 outperforms a traditional thermal transfer pad with a flat surface by better regulating skin temperature. This experiment tested performance of a thermal transfer pad used for cooling (where the pad is colder than the skin, and heat flows from the skin to the pad); the inventors expect that results will be similar when a pad is used for heating, and heat flows from the pad to the skin. In this experiment, a gel pack to simulate skin tissue was heated to 80 degrees F. An embodiment of the invention with profile 1101 of FIG. 11 was cooled in a freezer. A thermocouple was placed on the top surface of the simulated skin tissue, and then the frozen thermal pack was placed on top of the simulated skin tissue, with the thermocouple sandwiched between the simulated skin tissue and the profiled surface of the thermal pack. The temperature 1201 of the thermocouple was measured over time. The results were temperature trajectory 1211. For comparison, the flat (opposite) surface of the thermal pack, simulating an existing traditional thermal pack, was used in the same experiment, resulting in temperature trajectory 1210. This experiment clearly demonstrates that the profiled surface results in a much more stable temperature trajectory 1211, and that the temperature using a flat surface plunges very rapidly at the beginning, compared to a relatively modest temperature drop for the profiled surface. The inventors repeated this experiment with variations on profile 1101 and obtained similar results for a variety of profiled surfaces.

While the invention herein disclosed has been described by means of specific embodiments and applications thereof, numerous modifications and variations could be made thereto by those skilled in the art without departing from the scope of the invention set forth in the claims. For example, embodiments of the invention may be paired with temperature sensors, garments or sleeves or other couplers to facilitate use of embodiments of the invention.

What is claimed is:

1. A thermal transfer pad with a profiled surface to regulate skin temperature, comprising:
a thermal pad configured to be heated or cooled, the thermal pad having an application surface configured to be placed facing skin of a user; and,
protrusions extending from the application surface;
wherein:
each protrusion of the protrusions has a tip that is configured to be placed against the skin;
the tip of each protrusion has a height h comprising a maximum distance between the tip and the application surface;
the protrusions comprise a thermally conductive material configured to transfer heat between the thermal pad and the skin;
the application surface is partitioned into:
conductive zones from which protrusions protrude, and
air channel zones from which no protrusions protrude;
the air channel zones and do not contact the skin when tips of the protrusions are placed against the skin;
the protrusions are configured to support the skin at the tips of the protrusions while maintaining the air channel zones out of contact with the skin during therapeutic application of the thermal transfer pad;
the height h of the protrusions is between 1 mm and 6 mm, inclusive;
the height h of the protrusions and spacing between adjacent protrusions are cooperatively selected to maintain the air channel zones out of contact with the skin by avoiding deformation of the skin into the air channel zones and by avoiding collapse of the protrusions during therapeutic application of the thermal transfer pad;
the protrusions have an effective transfer surface that is an orthogonal projection of tips of the protrusions onto the application surface; and
a surface area of the effective transfer surface comprises between 5% and 40%, inclusive, of a surface area of the application surface;
wherein the protrusion tips define conductive contact regions between the thermal transfer pad and the skin, and the air channel zones define recessed non-contact air channel zones that reduce conductive heat transfer through the application surface.

2. The thermal transfer pad with a profiled surface to regulate skin temperature of claim 1, wherein the thermal transfer pad is configured to transfer heat from the skin to the thermal transfer pad.

3. The thermal transfer pad with a profiled surface to regulate skin temperature of claim 1, wherein the thermal transfer pad is configured to transfer heat from the thermal transfer pad to the skin.

4. The thermal transfer pad with a profiled surface to regulate skin temperature of claim 1, wherein:
in each square subregion of the application surface having an area of 16 square centimeters, a surface area of an intersection of the effective transfer surface with said each square subregion comprises between 5% and 40%, inclusive, of a surface area of said each square subregion;
wherein conductive contact regions are distributed across the application surface to maintain substantially uniform thermal transfer characteristics over the application surface.

5. The thermal transfer pad with a profiled surface to regulate skin temperature of claim 1, wherein the tips of the protrusions are substantially flat.

6. The thermal transfer pad with a profiled surface to regulate skin temperature of claim 1, wherein:
the protrusions comprise ridges;
each ridge of the ridges has a width between 0.5 mm and 10.0 mm, inclusive; and,
each air channel zone between adjacent ridges has a width less than or equal to three times the maximum distance between the ridge tips and the application surface, wherein deformation of the skin into the air channel zones is inhibited during therapeutic application.

7. The thermal transfer pad with a profiled surface to regulate skin temperature of claim 1, wherein:
the protrusions comprise pillars; and,
an orthogonal projection of the tip of each pillar of the pillars onto the application surface has an area between 1 square millimeter and 100 square millimeters, inclusive.

8. The thermal transfer pad with a profiled surface to regulate skin temperature of claim 1, wherein the protrusions have a hardness between 0 (Shore D) and 80 (Shore D), inclusive.

9. The thermal transfer pad with a profiled surface to regulate skin temperature of claim 1, wherein the protrusions comprise a compliant material.

10. The thermal transfer pad with a profiled surface to regulate skin temperature of claim 9, wherein the compliant material comprises an elastomer.

11. The thermal transfer pad with a profiled surface to regulate skin temperature of claim 10, wherein said elastomer comprises silicone rubber.

12. The thermal transfer pad with a profiled surface to regulate skin temperature of claim 1, wherein the protrusions comprise a material with thermal conductivity of between 0.14 W/m-K and 4.3 W/m-K, inclusive. and wherein the thermal conductivity contributes to thermal transfer through the protrusion tips while the recessed non-contact air channel zones reduce conductive heat transfer.

13. The thermal transfer pad with a profiled surface to regulate skin temperature of claim 1, wherein:
the protrusions have said height h;
the application surface has a total surface area S;
the effective transfer surface has a total surface area A; and,
a product (A/S)(1/h) is between 0.01 mm⁻¹ and 0.40 mm⁻¹, inclusive., and
wherein said product defines a thermal transfer geometry that maintains conductive contact through the protrusion tips while maintaining recessed non-contact air channel zones between adjacent protrusions during therapeutic application of the thermal transfer pad.

14. The thermal transfer pad with a profiled surface to regulate skin temperature of claim 1, wherein the thermal pad is filled with a liquid material.

15. The thermal transfer pad with a profiled surface to regulate skin temperature of claim 14, wherein the liquid material comprises a gel.

16. The thermal transfer pad with a profiled surface to regulate skin temperature of claim 14, wherein the liquid material has a freezing point at or below −20 degrees Fahrenheit.

17. The thermal transfer pad with a profiled surface to regulate skin temperature of claim 1, wherein the thermal pad is filled with a solid material.

18. The thermal transfer pad with a profiled surface to regulate skin temperature of claim 17, wherein the solid material comprises an aggregate.

19. The thermal transfer pad with a profiled surface to regulate skin temperature of claim 1, wherein the profiled surface occupies between 10% and 100% of a total surface area of a top and a bottom of the thermal transfer pad and wherein the profiled surface defines a thermal regulation region configured to contact the user while remaining portions of the thermal transfer pad comprise substantially flat surfaces.

20. A thermal transfer pad with a profiled surface to regulate skin temperature, comprising:
a thermal pad configured to be heated or cooled, the thermal pad having an application surface configured to be placed facing skin of a user; and,
protrusions extending from the application surface;
wherein:
each protrusion of the protrusions has a tip that is configured to be placed against the skin;
the tip of each protrusion has a height h comprising a maximum distance between the tip and the application surface;
the protrusions comprise a thermally conductive material configured to transfer heat between the thermal pad and the skin;
the thermally conductive material has a thermal conductivity of between 0.14 W/m-K and 4.3 W/m-K, inclusive;
the application surface is partitioned into:
conductive zones from which protrusions protrude; and
air channel zones from which no protrusions protrude;
the air channel zones are recessed relative to the tips of the protrusions and do not contact the skin when the tips of the protrusions are placed against the skin;
the protrusions
are configured to support the skin at the tips of the protrusions while maintaining the recessed air channel zones out of contact with the skin during therapeutic application of the thermal transfer pad;
the protrusions comprise ridges;
each ridge has a width between 0.5 mm and 10.0 mm, inclusive;
the height h of the protrusions is between 1 mm and 6 mm, inclusive;
the height h of the protrusions and spacing between adjacent ridges are cooperatively selected to maintain the recessed air channel zones out of contact with the skin by avoiding deformation of the skin into the air channel zones and by avoiding collapse of the protrusions during therapeutic application of the thermal transfer pad;
each air channel zone between adjacent ridges has a width less than or equal to three times the maximum distance between the ridge tips and the application surface;
